(19) United States
(12) Patent Application Publication
(10) Pub. No.: US 2021/0361211 A1
(43) Pub. Date: Nov. 25, 2021

(54) MEASURING DEVICE AND MEASURING SYSTEM

(71) Applicant: ISHIDA CO., LTD., Kyoto-shi, Kyoto (JP)

(72) Inventors: Taku TERAMOTO, Kyoto-shi, Kyoto (JP); Makoto NAKATANI, Kyoto-shi, Kyoto (JP); Yoshito KUNISAKI, Kyoto-shi, Kyoto (JP)

(21) Appl. No.: 17/275,575

(22) PCT Filed: Sep. 2, 2019
(86) PCT No.: PCT/JP2019/034479

§ 371 Date: Mar. 11, 2021

(30) Foreign Application Priority Data
Sep. 14, 2018 (JP) 2018-172949

(51) Int. Cl. A61B 5/20; G01G 17/04; G01G 23/42; A61B 5/00

(52) U.S. Cl. CPC A61B 5/20; A61B 5/742; G01G 23/42; G01G 17/04

(57) ABSTRACT

A measuring device includes a weighing unit configured to measure a mass value of a urine bag; a control unit configured to calculate at least one of a voided volume and a urinary output per unit time on the basis of the mass value weighed by the weighing unit; a communication unit configured to transmit urinary output information related to at least one of the voided volume and the urinary output per unit time calculated by the control unit; and a first battery configured to supply power to the weighing unit, the control unit, and the communication unit.

### TECHNICAL FIELD

The present invention relates to a measuring device and a measuring system.

### BACKGROUND ART

In hospitals and the like, urine bags are used for patients having difficulty in urinating by themselves after surgery with general anesthesia, or the like. A urine bag stores urine discharged from a patient. A measuring device measuring a urinary output stored in this urine bag is known (for example, refer to Patent Literature 1). In the measuring device, a urinary output is measured, and a display unit displays the measured urinary output.

### CITATION LIST

### Patent Literature

[Patent Literature 1] Japanese Unexamined Patent Publication No. 2012-105947

### SUMMARY OF INVENTION

### Technical Problem

In measuring devices in the related art, a urinary output is displayed in a display unit. For this reason, a staff member of a hospital, such as a nurse, regularly visits a hospital room, checks a urinary output displayed in the display unit, discharges urine from a urine bag to a graduated cylinder, and weighs the urine, thereby recording the amount of urine in a management sheet or the like. Accordingly, for example, the physical condition or the like of a patient after surgery is managed, or timings of taking urine from a urine bag are adjusted or the like. There is a need for a staff member to regularly visit a hospital room to perform work of checking a urine bag, and a burden may be placed on a staff member. Particularly, during work of checking at night having fewer staff members and being work performed in the darkness, a burden on a staff member increases.

A measuring device is normally attached to a bed. For example, when a patient temporarily moves from a bed, a measuring device needs to move in accordance with movement of the patient. In this case, work of securing a power source outlet at a destination and attaching a power source adapter or the like for supplying power to the measuring device occurs.

An object of an aspect of the present invention is to provide a measuring device and a measuring system capable of achieving reduction of a burden.

### Solution to Problem

According to an aspect of the present invention, there is provided a measuring device acquiring information related to urine stored in a urine bag. The measuring device includes a weighing unit configured to measure a mass value of the urine bag; a calculation unit configured to calculate at least one of a voided volume and a urinary output per unit time on the basis of the mass value weighed by the weighing unit; a transmission unit configured to transmit urinary output information related to at least one of the voided volume and the urinary output per unit time calculated by the calculation unit; and a first battery configured to supply power to the weighing unit, the calculation unit, and the transmission unit.

In the measuring device according to the aspect of the present invention, at least one of the voided volume and the urinary output per unit time is calculated, and urinary output information is transmitted. Accordingly, for example, a different terminal can acquire urinary output information by receiving the urinary output information. In this manner, in the measuring device, even if a staff member does not visit a hospital room, the voided volume or the like can be checked on the basis of urinary output information in the different terminal. Therefore, there is no need for a staff member to perform work of visual checking. For this reason, in the measuring device, reduction of a burden on a staff member can be achieved. In addition, it is possible to reduce a burden on a staff member performing checking at night, that is, performing work in the darkness with less manpower.

The measuring device includes the first battery supplying power to the weighing unit, the calculation unit, and the transmission unit. For this reason, in the measuring device, for example, when a patient temporarily moves, each unit can be driven even if a power source outlet is not secured. Therefore, a burden on a staff member can be reduced. In addition, in the measuring device, power can be supplied to each unit while the measuring device is moving or the like. Therefore, in the measuring device, urinary output information can be transmitted even while the measuring device is moving or the like.

According to the embodiment, the measuring device may further include a display unit configured to be able to display the urinary output information, a control unit configured to control contents of display of the display unit, and an acquisition unit configured to acquire a state of the first battery. The control unit may cause the display unit to display the state of the first battery acquired by the acquisition unit. In this configuration, the state (residual quantity or the like) of the first battery can be checked in the display unit.

According to the embodiment, the control unit may cause the display unit to display that a residual quantity of the first battery has decreased when the residual quantity becomes equal to or smaller than a threshold. In this configuration, when the residual quantity of the first battery has decreased, it is possible to notify that the first battery needs to be charged.

According to the embodiment, the measuring device may further include a determination unit configured to determine anuria, oliguria, and polyuria on the basis of the urinary output information and a threshold set in advance. The transmission unit may transmit notification information when the determination unit determines anuria, oliguria, or polyuria. In this configuration, a staff member can be notified of anuria, oliguria, or polyuria. In addition, since the measuring device includes a battery, notification information can be transmitted even while the measuring device is moving or the like.

According to the embodiment, the measuring device may further include a second battery configured to supply at least power for memory back-up when power is not supplied from the first battery. In this configuration, even when power is not supplied from a power source unit and the first battery, setting and the like in the measuring device, such as setting of a timepiece, measurement results, and measurement history can be maintained by the second battery.

According to another aspect of the present invention, there is provided a measuring system including the measuring device described above, and a management device configured to acquire information related to urine acquired by the measuring device.

In the measuring system according to the aspect of the present invention, the measuring device calculates at least one of the voided volume and the urinary output per unit time and transmits urinary output information. Accordingly, the management device can acquire urinary output information by receiving the urinary output information. In this manner, in the measuring system, even if a staff member does not visit a hospital room, the voided volume or the like can be checked on the basis of urinary output information in the management device. Therefore, there is no need for a staff member to perform work of visual checking. For this reason, in the measuring system, reduction of a burden on a staff member can be achieved. In addition, it is possible to reduce a burden on a staff member performing checking at night, that is, performing work in the darkness with less manpower.

The measuring device includes a battery supplying power to the weighing unit, the calculation unit, and the transmission unit when power is not supplied from the power source unit. For this reason, in the measuring device, for example, when a patient temporarily moves, each unit can be driven even if a power source outlet is not secured. Therefore, a burden on a staff member can be reduced. In addition, in the measuring device, power can be supplied to each unit while the measuring device is moving or the like. Therefore, in the measuring system, even while the measuring device is moving or the like, urinary output information can be transmitted and urinary output information can be acquired in the management device.

### Advantageous Effects of Invention

According to the aspects of the present invention, reduction of a burden can be achieved.

### BRIEF DESCRIPTION OF DRAWINGS

FIG. 1 is a view illustrating a measuring system according to an embodiment.

FIG. 2 is a perspective view illustrating a measuring device.

FIG. 3 is another perspective view illustrating the measuring device.

FIG. 4 is a view illustrating a configuration of the measuring device of the measuring system illustrated in FIG. 1.

FIG. 5 is a view illustrating a configuration of a weighing unit.

FIG. 6 is a view illustrating an example of a screen displayed in a touch panel display.

FIG. 7 is a view illustrating another example of a screen displayed in the touch panel display.

FIG. 8 is a view illustrating another example of a screen displayed in the touch panel display.

FIG. 9 is a view illustrating another example of a screen displayed in the touch panel display.

FIG. 10 is a view illustrating another example of a screen displayed in the touch panel display.

FIG. 11 is a view illustrating another example of a screen displayed in the touch panel display.

FIG. 12 is a view illustrating another example of a screen displayed in the touch panel display.

FIG. 13 is a view illustrating a configuration of a management device.

FIG. 14 is a view illustrating an example of a screen displayed in a display unit.

FIG. 15 is a view illustrating another example of a screen displayed in the display unit.

FIG. 16 is a perspective view illustrating a measuring device according to a different embodiment.

FIG. 17 is a perspective view illustrating the measuring device according to the different embodiment.

FIG. 18 is a front view of the measuring device according to the different embodiment.

### DESCRIPTION OF EMBODIMENTS

Hereinafter, preferable embodiments of the present invention will be described in detail with reference to the accompanying drawings. In description of the drawings, the same reference signs are applied to elements which are the same or corresponding, and duplicate description will be omitted.

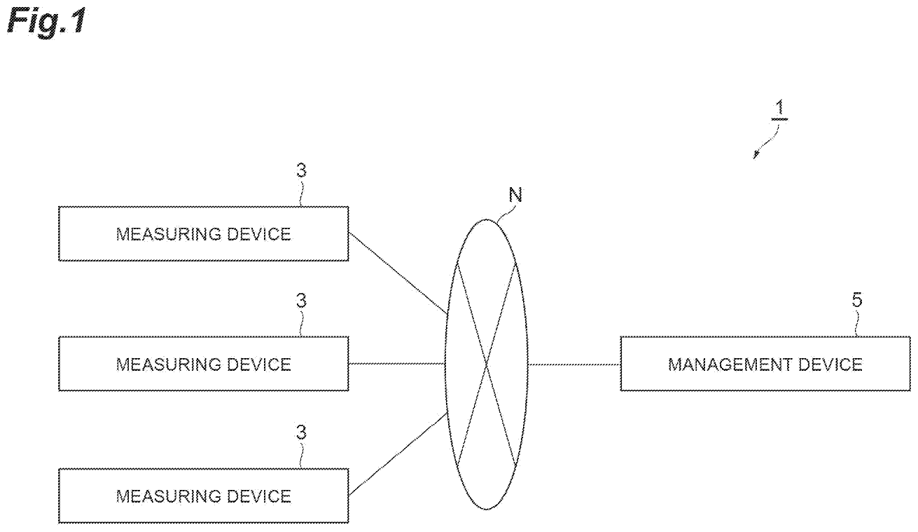

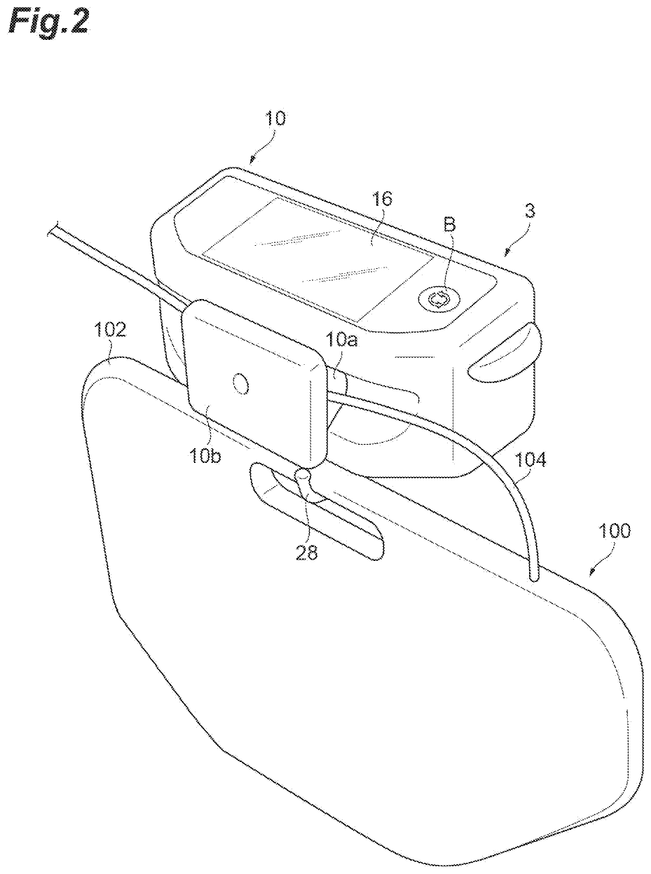

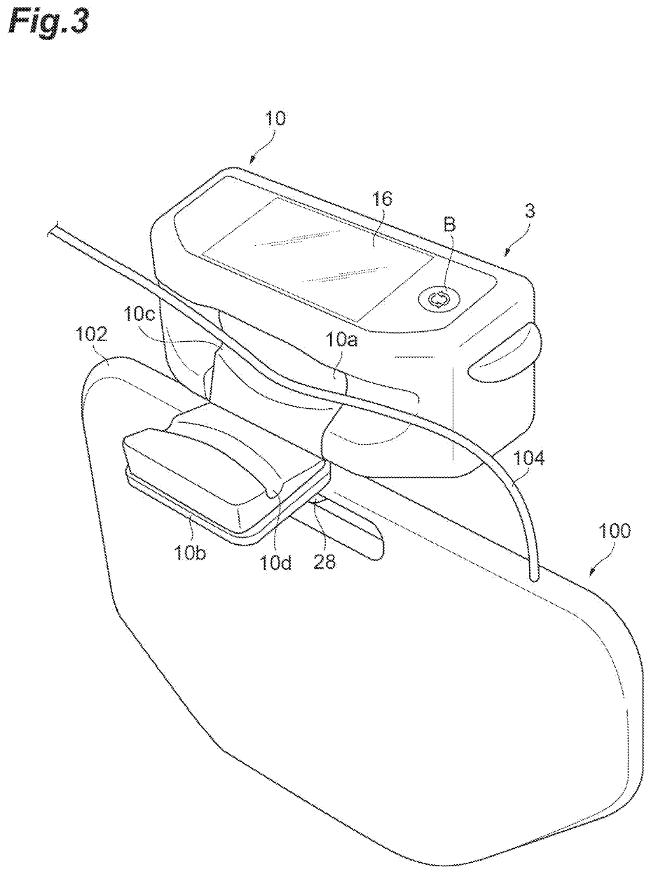

As illustrated in FIG. 1, a measuring system 1 includes measuring devices 3 and a management device 5. In the present embodiment, a plurality of measuring devices 3 are provided. The measuring devices 3 and the management device 5 can communicate with each other via a network N such as a wireless local area network (LAN). For example, the measuring system 1 manages patient's urine stored in a urine bag 100 (refer to FIGS. 2 and 3) in a hospital or the like.

The measuring device 3 acquires information related to urine stored in the urine bag 100. As illustrated in FIGS. 2 and 3, the urine bag 100 has a storage unit 102 storing urine, and a urine guide tube 104 for introducing urine into the storage unit 102. The urine bag 100 may be provided with a voidance tube for discharging urine stored in the storage unit 102. Various commercially available bags can be used as the urine bag 100.

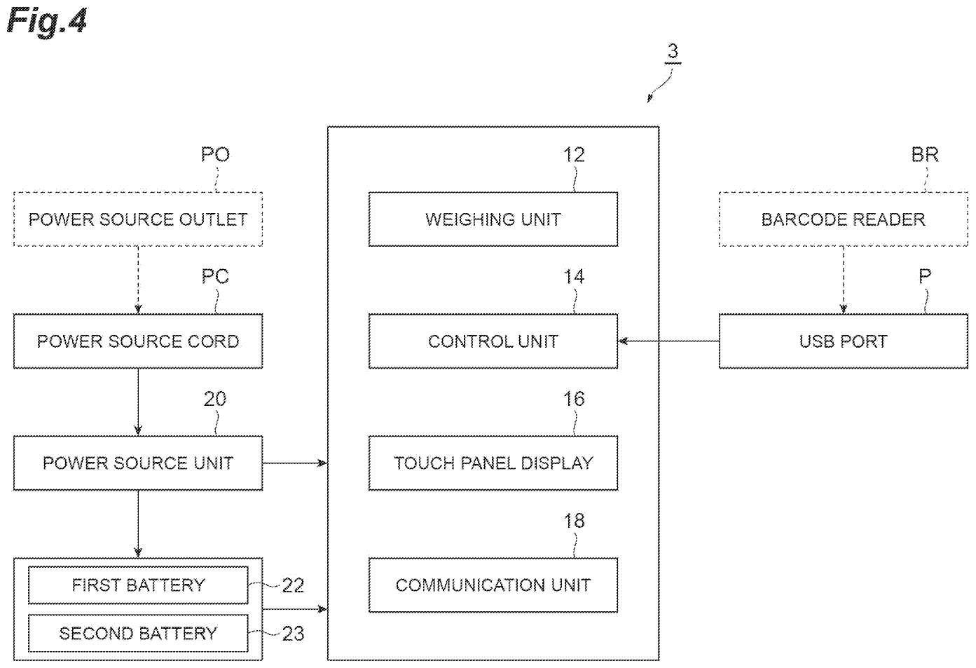

As illustrated in FIG. 4, the measuring device 3 has a housing 10 (refer to FIGS. 2 and 3), a weighing unit 12, a control unit (a calculation unit, an acquisition unit, and a determination unit) 14, a touch panel display (a display unit) 16, a communication unit (a transmission unit) 18, a power source unit 20, a first battery 22, and a second battery.

For example, the housing 10 is formed of an ABS resin or the like. As illustrated in FIGS. 2 and 3, the housing 10 has a holding portion 10a and a fixing portion 10b. The holding portion 10a is provided with a groove (a recessed portion) 10c in which the urine guide tube 104 is disposed. The fixing portion 10b is provided at a central portion of the holding portion 10a. The fixing portion 10b is provided in a swingable manner in the holding portion 10a. The fixing portion 10b is provided with a groove 10d in which the urine guide tube 104 is disposed. As illustrated in FIG. 3, the urine guide tube 104 can be attached to the holding portion 10a and the urine guide tube 104 can be detached from the holding portion 10a by opening the fixing portion 10b. As illustrated in FIG. 2, the urine guide tube 104 can be sandwiched between the groove 10c and the groove 10d and the urine guide tube 104 can be fixed to the holding portion 10a by closing the fixing portion 10b. The fixing portion 10b restricts movement of the urine guide tube 104 in the holding portion 10a.

As illustrated in FIG. 4, the housing 10 is provided with a measurement button B for starting measurement (refer to FIGS. 2 and 3) and a USB port P. In addition, the housing 10 is provided with a connection portion (not illustrated) to which a power source cord PC for supplying power to the measuring device 3 is connected.

A terminal to be connected to a connection portion of the measuring device 3 is provided at one end of the power source cord PC, and an outlet plug to be connected to a power source outlet PO is provided at the other end of the power source cord PC. When the outlet plug of the power source cord PC is connected to the power source outlet PO, the measuring device 3 may receive supply of power from the power source outlet PO. Power supplied from the power source outlet PO is supplied to the power source unit 20.

A barcode reader (a reading device) BR can be connected to the USB port P. The barcode reader BR reads barcodes including patient information. Patient information may include the name or the like of a patient. For example, a barcode is displayed in a band attached to a wrist of a patient. The barcode reader BR outputs read patient information to the control unit 14.

The housing 10 is provided with an attachment portion (not illustrated) for attaching the measuring device 3 to a bed, a drip infusion stand, or the like. For example, the attachment portion is disposed on a rear surface (a back surface) side of the housing 10. The attachment portion may be provided in a manner of being attachable/detachable with respect to the housing 10. The attachment portion can be switched in accordance with an attachment target (a bed, a drip infusion stand, or the like).

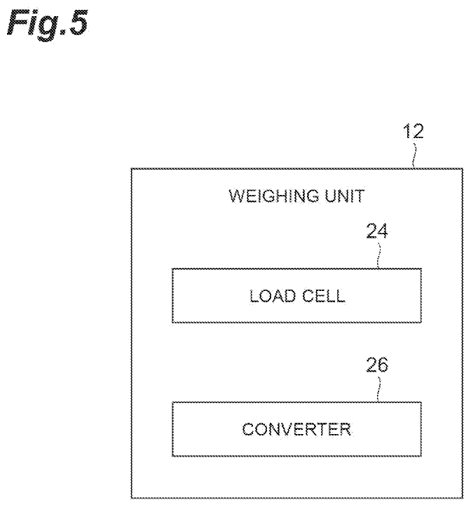

The weighing unit 12 measures the mass value of a urine bag. As illustrated in FIG. 5, the weighing unit 12 has a load cell 24 and a converter 26.

The load cell 24 is connected to the housing 10 and a hook 28. Specifically, in the load cell 24, one end thereof is fixed to the housing 10, and the hook 28 is provided at the other end thereof. For example, the hook 28 is formed of a resin. The hook 28 can move (swing) to a position where the urine bag 100 can be interlocked, that is, an interlock position (a position illustrated in FIG. 2) protruding from the housing 10, and an accommodation position of being accommodated inside the housing 10 when the urine bag 100 is not interlocked. The hook 28 may have any shape as long as the urine bag 100 can be held (the urine bag 100 does not fall oft) with the shape.

The load cell 24 detects the mass of the storage unit 102 of the urine bag 100 interlocked with the hook 28. The load cell 24 detects the mass of the storage unit 102 of the urine bag 100 and outputs weighing data (a mass value) regarding the mass to the converter 26. Weighing data output by the load cell 24 is an analog signal such as a voltage corresponding to the mass of an article to be weighed. The load cell 24 is configured to include an element measuring a distortion of a semiconductor pressure sensor or the like. The load cell 24 is not particularly limited, and various kinds of known load cells can be employed.

The converter 26 converts weighing data as an analog signal output from the load cell 24 into weighing data as a digital signal. The converter 26 outputs weighing data to the control unit 14.

The control unit 14 is configured to have a central processing unit (CPU), a read only memory (ROM), a random access memory (RAM), a hard disc drive (HDD), and the like. The control unit 14 receives an input from an operator and performs various kinds of setting of the measuring device 3. When a predetermined operation is performed in a standby screen D1 of the touch panel display 16, the control unit 14 causes the touch panel display 16 to display a setting screen.

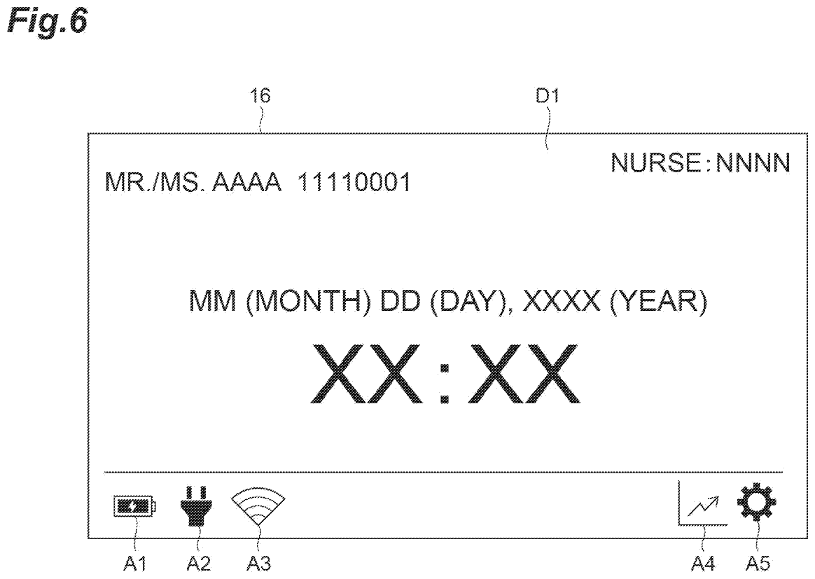

As illustrated in FIG. 6, the standby screen D1 displays “patient name”, “patient ID”, “nurse name”, “date and time”, “state icon”, “selection icon”, and the like. Regarding the “state icon”, for example, a residual quantity icon A1 indicating a state (residual quantity) of the first battery 22, an icon A2 indicating a connection state of the power source cord PC, and an icon A3 indicating a connection state (reception intensity) of a wireless LAN are displayed. Regarding the “selection icon”, a history icon A4 and a setting icon A5 are displayed. When the setting icon A5 is touched in the standby screen D1 displayed in the touch panel display 16, the control unit 14 causes the touch panel display 16 to display the setting screen.

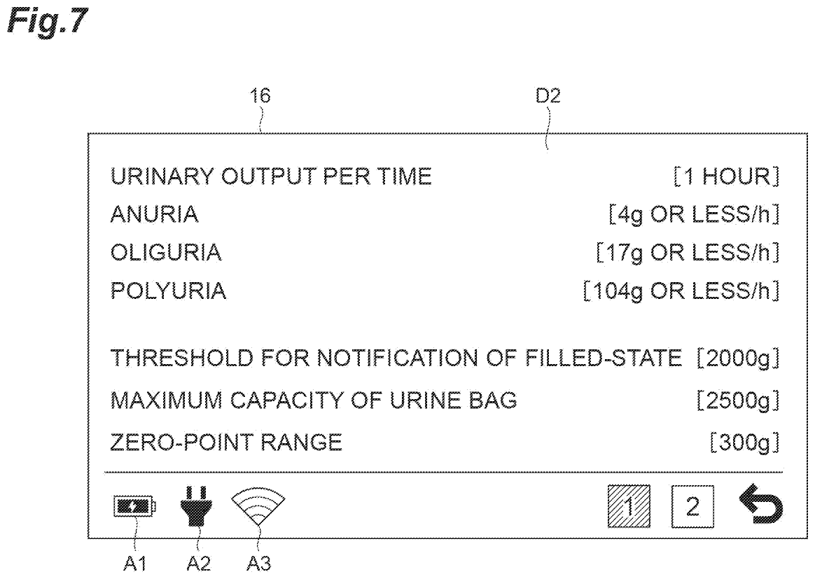

As illustrated in FIG. 7, for example, parameters can be set in a setting screen D2. For example, regarding parameter setting items, the setting screen D2 displays “urinary output per time”, “anuria”, “oliguria”, “polyuria”, “threshold for notification of filled-state”, “maximum capacity of urine bag”, “zero-point range”, and the like. For example, regarding the “urinary output per time”, “1 minute (g/min)”, “1 hour (g/hour)”, or “1 day (g/day)” can be set. Numerical values (thresholds) can be set for “anuria”, “oliguria”, “polyuria”, “threshold for a notification of filled-state”, “maximum capacity of urine bag”, and “zero-point range”. Numerical values for “anuria”, “oliguria”, and “polyuria” may be automatically calculated and displayed by inputting the body weight of a patient.

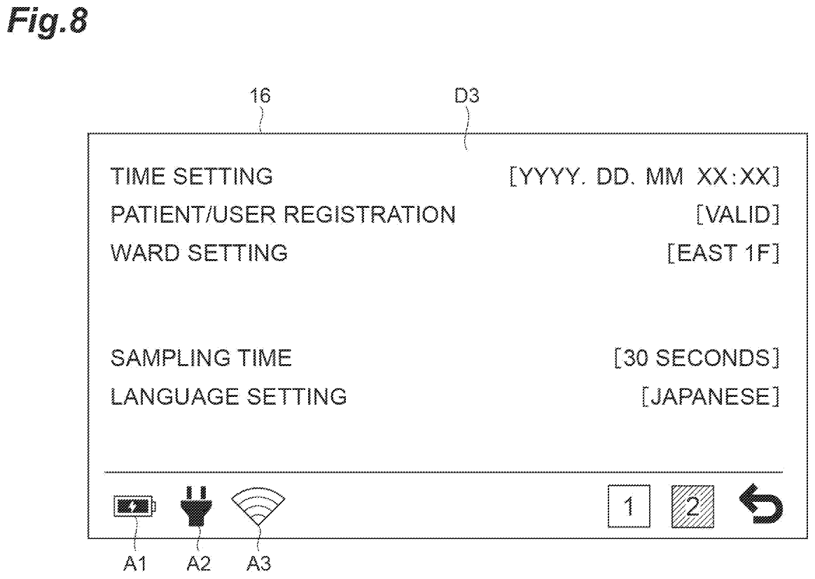

As illustrated in FIG. 8, for example, various kinds of setting can be performed in a setting screen D3. In the setting screen D2, “time setting”, “patient/user information”, “ward setting”, “sampling time”, and “language setting” can be set. Regarding the “language setting”, for example, Japanese, English, or the like can be selected.

In addition, for example, items regarding wireless connection can be set in another setting screen. For example, regarding setting items for wireless connection, the setting screen displays “ON/OFF of Wi-Fi”, “Wi-Fi SSID”, “Wi-Fi password”, “security authentication method”, “IP address”, “subnet mask”, “IP address of gateway”, “MAC address”, “2.4 GHz/5 GHz”, and the like. In the setting screen, communication according to any of the standards of IEEE802.11b/g/n/a (2.4 GHz/5 GHz) can be set.

The control unit 14 inputs patient information output from the barcode reader BR and registers the patient information. When a predetermined operation is performed by an operator for the item “patient/user registration” in the setting screen D2, the control unit 14 causes the barcode reader BR to start reading. When patient information output from the barcode reader BR is received, the control unit 14 manages the patient information and urinary output information (which will be described below) in association with each other.

The control unit 14 calculates a voided volume and a urinary output per unit time on the basis of the mass value weighed by the weighing unit 12. The control unit 14 calculates a weight from weighing data output from the converter 26 and calculates the weight (voided volume) [g] of urine by subtracting a tare weight (a weight including the urine bag 100 and the like) from the weight. A control unit 54 stores urinary output information indicating the calculated weight of urine in the HDD or the like. The control unit 14 calculates a urinary output [g] per unit time on the basis of the calculated weight of urine. The control unit 14 calculates a urinary output per unit time (1 hour or 1 day) in accordance with contents of setting. The control unit 14 stores the calculated urinary output per unit time in the HDD or the like.

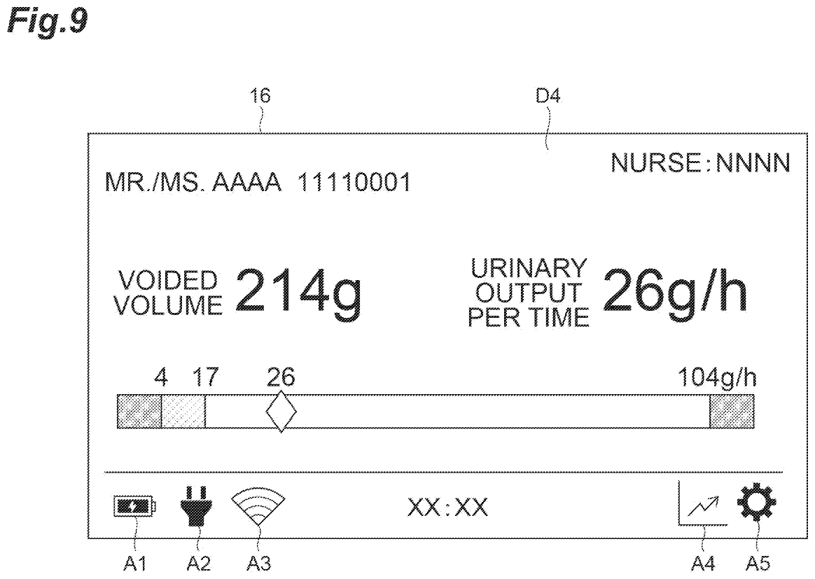

The control unit 14 causes the touch panel display 16 to display the voided volume and the urinary output per unit time. As illustrated in FIG. 9, the control unit 14 causes a measurement screen D4 to display a numerical value [g] for “voided volume” and a numerical value [g] for “urinary output per time”. The control unit 14 causes the measurement screen D4 to visually display current urinary output per unit times for anuria, oliguria, and polyuria. Specifically, the control unit 14 causes a bar in which parameters for anuria, oliguria, and polyuria are displayed to be displayed and causes a urinary output per unit time to be displayed on the bar.

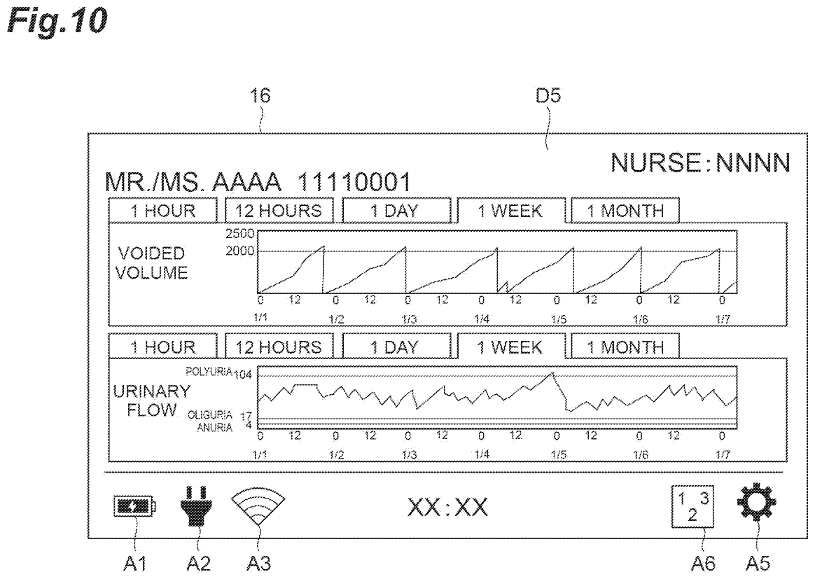

The control unit 14 causes the touch panel display 16 to display history of urinary outputs. When the history icon A4 is touched in the standby screen D1, the control unit 14 causes history of the amount of urine to be displayed. As illustrated in FIG. 10, in a history screen D5, history regarding “voided volume” and “urinary flow (urinary output per unit time)” is displayed in a graph. For example, the history can be displayed in units of “hour”, “12 hours”, “1 day”, “week”, and “1 month”. In the example illustrated in FIG. 10, history of 1 week is illustrated. When a measurement icon A6 is touched, the control unit 14 causes the touch panel display 16 to display the measurement screen D4 illustrated in FIG. 9.

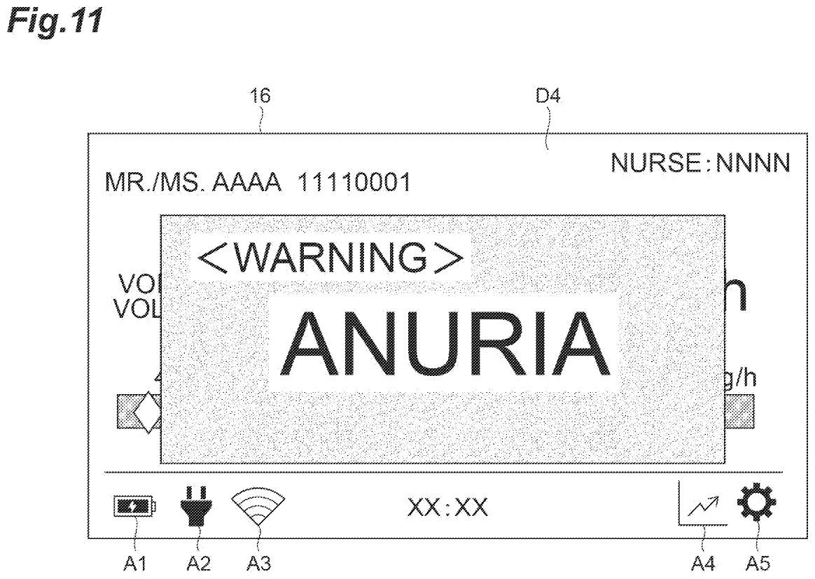

The control unit 14 determines anuria, oliguria, or polyuria on the basis of a urinary output per unit time. The control unit 14 determines anuria, oliguria, or polyuria on the basis of parameters set regarding anuria, oliguria, and polyuria set in advance. When the control unit 14 determines anuria, oliguria, or polyuria, the control unit 14 outputs notification information for causing the touch panel display 16 to display anuria, oliguria, or polyuria to the touch panel display 16. As illustrated in FIG. 11, for example, the control unit 14 causes the touch panel display 16 to display a warning pop-up. The control unit 14 may issue a notification using a buzzer or the like in addition to display in the touch panel display 16. When the voided volume becomes equal to or greater than the threshold for a notification of filled-state, the control unit 14 may output notification information for notifying that the urine bag 100 is in a filled-state to the touch panel display 16 on the basis of urinary output information.

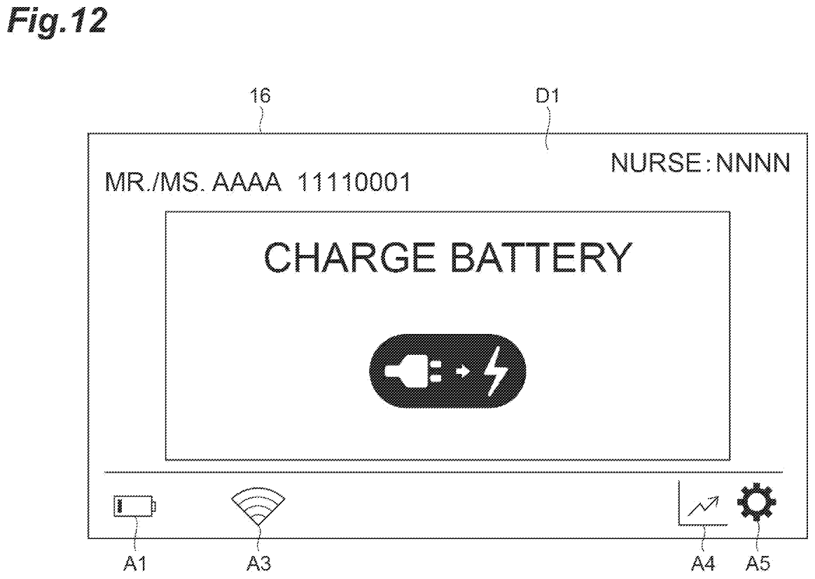

The control unit 14 acquires the state of the first battery 22. The control unit 14 acquires the residual quantity of the first battery 22. The control unit 14 controls display of the residual quantity icon A1 displayed in the touch panel display 16 in accordance with the residual quantity of the first battery 22. When the residual quantity of the first battery 22 becomes equal to or smaller than the threshold, the control unit 14 causes the touch panel display 16 to display that the residual quantity has decreased. As illustrated in FIG. 12, the control unit 14 causes the touch panel display 16 to display a pop-up indicating that residual quantity of the first battery 22 has decreased (prompting a user to perform charging). The control unit 14 may issue a notification using a buzzer or the like in addition to display in the touch panel display 16.

The control unit 14 outputs urinary output information to the communication unit 18. A timing when the control unit 14 outputs urinary output information to the communication unit 18 may be a timing when urinary output information is calculated or may be a predetermined time interval. The control unit 14 causes the communication unit 18 to transmit urinary output information.

The touch panel display 16 can display texts, images, or the like and receives an input of an operator. In the present embodiment, the touch panel display 16 can perform display in colors (256 or more colors). Contents of display in the touch panel display 16 can be controlled by the control unit 14.

The communication unit 18 is a wireless LAN module for wirelessly transmitting urinary output information. When urinary output information is output from the control unit 14, the communication unit 18 wirelessly transmits urinary output information in association with a unique identification code of the measuring device 3. Regarding a communication method of the communication unit 18, various kinds of known wireless communication methods (IEEE802.11b/g/n/a) can be utilized. When notification information is output from the control unit 14, the communication unit 18 transmits notification information to the management device 5.

The power source unit 20 supplies power to each unit of the measuring device 3. Specifically, the power source unit 20 supplies power to the weighing unit 12, the control unit 14, the touch panel display 16, and the first battery 22. The power source unit 20 receives supply of power from the power source outlet PO via the power source cord PC.

When power is not supplied from the power source unit 20, the first battery 22 supplies power to the weighing unit 12, the control unit 14, and the touch panel display 16. For example, the first battery 22 is a lithium-ion battery. The first battery 22 is charged upon reception of supply of power from the power source unit 20. The first battery 22 is charged by a charging device (not illustrated) connected to the power source unit 20. When power is no longer supplied from the power source unit 20, the first battery 22 starts supply of power. The first battery 22 may be attachable/detachable with respect to the housing 10. In this case, the first battery 22 can be charged by a charger provided in a nurse station or the like.

The second battery 23 supplies power for memory back-up. For example, the second battery 23 is a lithium-ion battery. The second battery 23 may have a smaller capacity than the first battery 22. When power is not supplied from the power source unit 20 and the first battery 22 is detached from the housing 10 or when the residual quantity of the first battery 22 has decreased, the second battery 23 supplies power to at least the control unit 14. The second battery 23 is charged upon reception of supply of power from the power source unit 20.

The management device 5 functions as a host device integrally managing the measuring device 3. The management device 5 manages data measured by the measuring device 3. For example, the management device 5 may be constituted of a dedicated computer, or a commercially available personal computer in which software (a program for electronic medical records) is installed may be used.

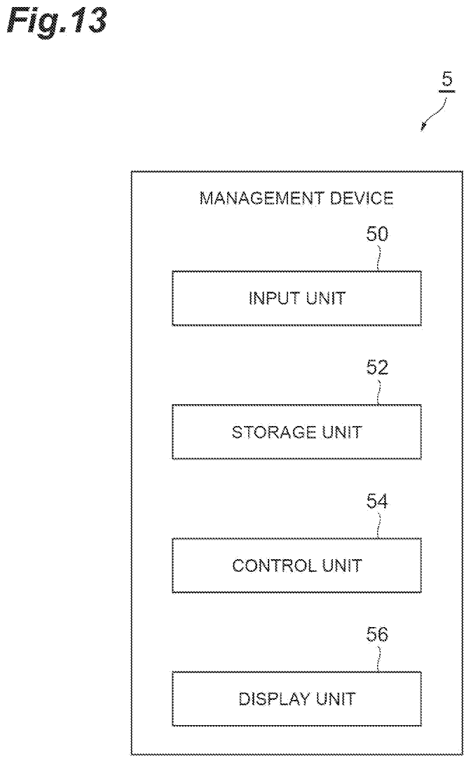

As illustrated in FIG. 13, the management device 5 has a communication unit 50, a storage unit 52, the control unit 54, and a display unit 56.

The communication unit 50 is a wireless LAN module for wirelessly receiving urinary output information. The communication unit 50 outputs received urinary output information and an identification code to the storage unit 52.

The storage unit 52 is configured to have a ROM, a RAM, an HDD, and the like. The storage unit 52 stores urinary output information received by the communication unit 50 in association with patient information.

The control unit 54 is configured to have a CPU and the like. For example, the control unit 54 controls display of the display unit 56 in accordance with an operation of an operation unit (not illustrated) such as a keyboard. For example, the display unit 56 is a display of a computer. For example, when an operation of requiring display of urinary output patient information is received, the control unit 54 acquires urinary output information from the storage unit 52 and causes the display unit 56 to display the acquired urinary output information.

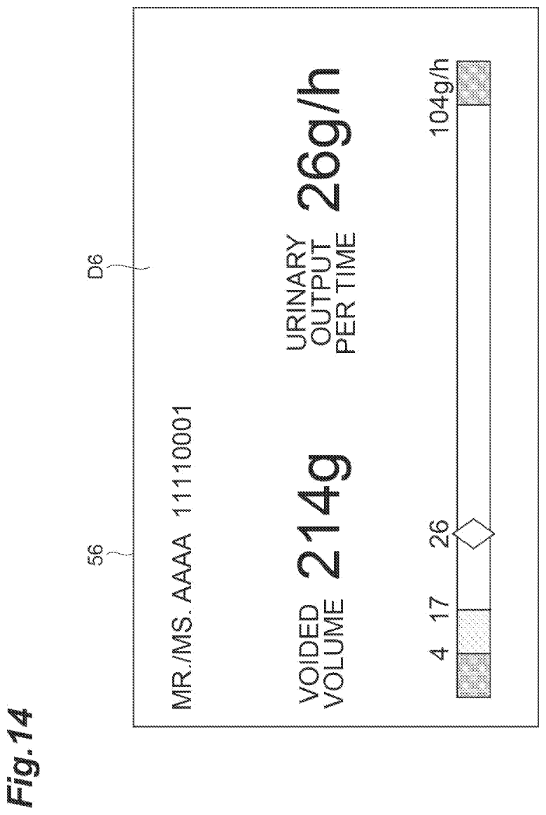

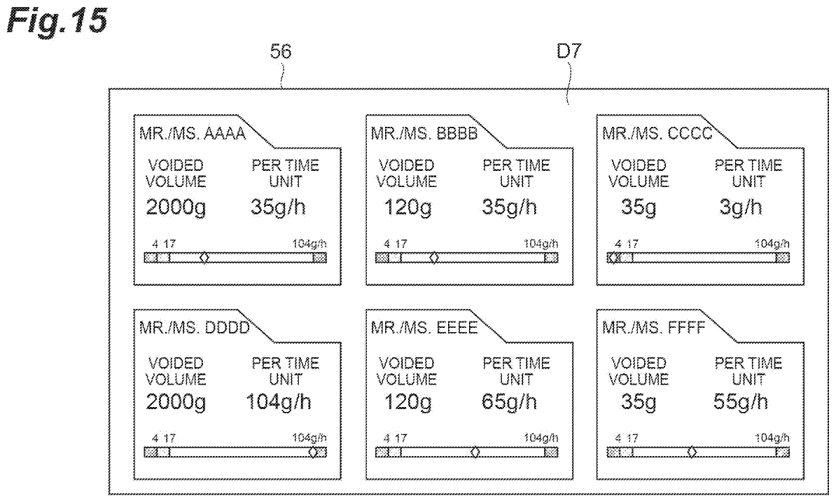

As illustrated in FIG. 14, for example, “voided volume” and “urinary output per time” are displayed for each patient in a display screen D6 of the display unit 56. The display screen D6 displays current urinary output per unit times for anuria, oliguria, and polyuria in a bar. In addition, as illustrated in FIG. 15, data of each patient may be displayed in a table in a display screen D7 of the display unit 56.

The control unit 54 may receive an input of patient information and may transmit patient information to the measuring device 3. In this configuration, the measuring device 3 may receive patient information transmitted from the management device 5 using the communication unit 18 and may register the patient information. In addition, when patient information read by the barcode reader BR coincides with patient information transmitted from the management device 5, the measuring device 3 may register the patient information.

Subsequently, a method of utilizing the measuring system 1 will be described. In order to use the measuring device 3, first, the urine bag 100 is set to the measuring device 3. Subsequently, a power source button (not illustrated) of the measuring device 3 is pressed. Accordingly, the power source of the measuring device 3 is turned on. When the measurement button B is pressed, the measuring device 3 starts measurement. When the measurement button B is pressed, the measuring device 3 performs zero-point calibration. At this time, when equal to or more than a predetermined amount of urine is stored in the urine bag 100, that is, when urine of an amount equal to or more than “zero-point range” is stored in the urine bag 100, the measuring device 3 does not perform zero-point calibration. The measuring device 3 starts to measure a urinary output after zero-point calibration. When measurement of a urinary output starts, the measuring device 3 transmits urinary output information to the management device 5. When the urinary output information is received, the management device 5 inputs a value based on the urinary output information in electronic medical records.

As described above, in the measuring system 1 according to the present embodiment, the measuring device 3 calculates the voided volume and the urinary output per unit time and transmits urinary output information to the management device 5. Accordingly, the management device 5 can acquire urinary output information by receiving the urinary output information. In this manner, in the measuring system 1, even if a staff member does not visit a hospital room, the voided volume or the like can be checked on the basis of urinary output information in the management device 5. Therefore, there is no need for a staff member to perform work of visual checking. For this reason, in the measuring system 1 including the measuring device 3, reduction of a burden on a staff member can be achieved. In addition, it is possible to reduce a burden on a staff member performing checking at night, that is, performing work in the darkness with less manpower.

The measuring device 3 includes the first battery 22 supplying power to the weighing unit 12, the control unit 14, and the communication unit 18 when power is not supplied from the power source unit 20. For this reason, in the measuring device 3, for example, when a patient temporarily moves, each unit can be driven even if the power source outlet PO is not secured. Therefore, a burden on a staff member can be reduced. In addition, in the measuring device 3, power can be supplied to each of the units (the weighing unit 12, the control unit 14, and the communication unit 18) while the measuring device 3 is moving or the like. Therefore, in the measuring device 3, urinary output information can be transmitted to the management device 5 even while the measuring device is moving or the like.

In the measuring system 1 according to the present embodiment, the measuring device 3 includes the touch panel display 16 capable of displaying urinary output information. The control unit 14 acquires the state of the first battery 22 and causes the touch panel display 16 to display the acquired state of the first battery 22. In this configuration, the state (residual quantity or the like) of the first battery 22 can be checked in the touch panel display 16.

In the measuring system 1 according to the present embodiment, the control unit 14 of the measuring device 3 causes the touch panel display 16 to display that the residual quantity has decreased when the residual quantity of the first battery 22 becomes equal to or smaller than the threshold. In this configuration, when the residual quantity of the first battery 22 has decreased, it is possible to notify that the first battery 22 needs to be charged.

In the measuring system 1 according to the present embodiment, the control unit 14 of the measuring device 3 determines anuria, oliguria, and polyuria on the basis of urinary output information and a threshold set in advance and outputs notification information when it determines anuria, oliguria, or polyuria. In this configuration, a staff member can be notified of anuria, oliguria, or polyuria.

In the measuring system 1 according to the present embodiment, the measuring device 3 includes the second battery 23 supplying at least power for memory back-up when power is not supplied from the power source unit 20 and the first battery 22. In this configuration, even when power is not supplied from the power source unit 20 and the first battery 22, setting and the like in the measuring device 3, such as setting of a timepiece, measurement results, and measurement history can be maintained by the second battery 23.

Hereinabove, an embodiment of the present invention has been described. However, the present invention is not necessarily limited to the embodiment described above, and various changes can be made within a range not departing from the gist thereof.

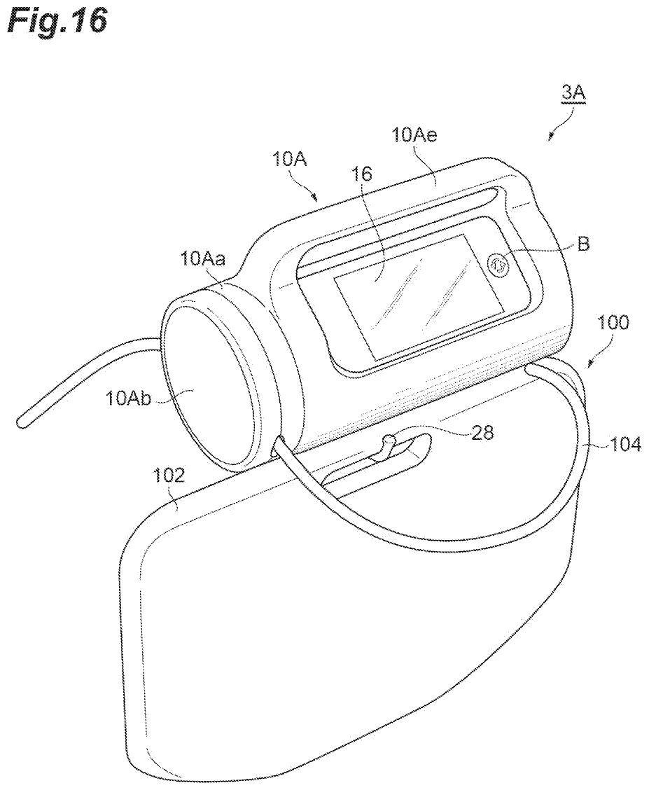

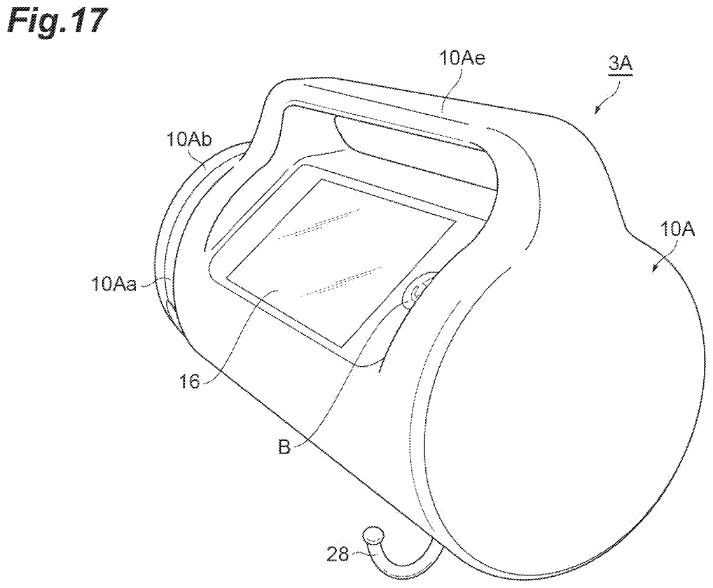

In the foregoing embodiment, a form in which the housing 10 of the measuring device 3 has a configuration illustrated in FIG. 1 has been described as an example. However, the configuration of a housing is not limited thereto. As illustrated in FIGS. 16, 17, and 18, a measuring device 3A has a housing 10A and the touch panel display 16. Similar to the measuring device 3, the measuring device 3A further has the weighing unit 12, the control unit 14, the communication unit 18, the power source unit 20, the first battery 22, and the second battery 23.

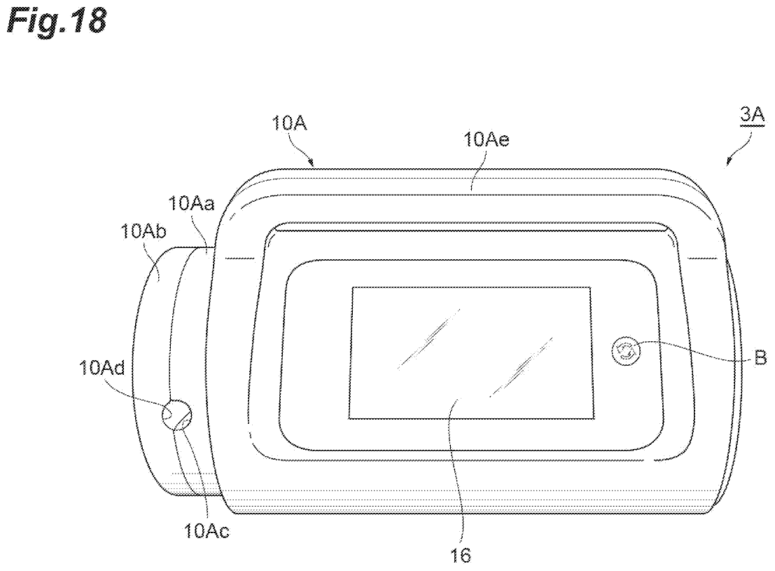

For example, the housing 10A is formed of an ABS resin or the like. The housing 10A exhibits a cylindrical shape. The housing 10A has a holding portion 10Aa and a fixing portion 10Ab. The holding portion 10Aa is provided with a groove 10Ac in which the urine guide tube 104 is disposed (refer to FIG. 18). The fixing portion 10Ab is provided at a position facing the holding portion 10Aa. The fixing portion 10Ab is provided in a swingable manner in the holding portion 10Aa. The fixing portion 10Ab is provided with a groove 10Ad in which the urine guide tube 104 is disposed (refer to FIG. 18). The urine guide tube 104 can be attached to the holding portion 10Aa and the urine guide tube 104 can be detached from the holding portion 10Aa by opening the fixing portion 10Ab. The urine guide tube 104 can be sandwiched between the groove 10Ac and the groove 10Ad and the urine guide tube 104 can be fixed to the holding portion 10Aa by closing the fixing portion 10Ab. The fixing portion 10Ab restricts movement of the urine guide tube 104 in the holding portion 10Aa. The housing 10A is provided with a grip 10Ae.

In the foregoing embodiments, a form in which the holding portion 10a or 10Aa and the fixing portion 10b are provided in the housing 10 or 10A of the measuring device 3 or 3A has been described as an example. However, the holding portion 10a and the fixing portion 10b may not be provided in the housing 10.

In the foregoing embodiments, a form in which the barcode reader BR is connected to the USB port P provided in the housing 10 or 10A of the measuring device 3 or 3A and a barcode including patient information is read by the barcode reader BR has been described as an example. However, a method of acquiring patient information is not limited thereto. For example, when patient information is included in a two-dimensional code, a device capable of reading a two-dimensional code may be connected to the USB port P. When patient information is included in an IC card in a non-contact manner, a device capable of reading an IC card in a non-contact manner may be connected to the USB port P.

In the foregoing embodiments, a form in which the display unit is the touch panel display 16 in the measuring device 3 or 3A has been described as an example. However, the measuring device 3 or 3A may include a display serving as a display unit, keys (buttons) serving as an operation unit, and the like.

In the foregoing embodiments, a form in which the first battery 22 and the second battery 23 are lithium-ion batteries has been described as an example. However, the first battery 22 and the second battery 23 may be different secondary batteries such as lead storage batteries. In the present invention, a secondary battery includes no capacitor.

In the foregoing embodiments, a form in which the control unit 14 of the measuring device 3 or 3A calculates the voided volume and the urinary output per unit time in [g] has been described as an example. However, the control unit 14 may calculate the voided volume and the urinary output per unit time in [ml]. In addition, the measuring device 3 may calculate at least one of the voided volume and the urinary output per unit time.

In addition to the foregoing embodiments, the measuring device 3 or 3A may acquire information regarding urine in addition to a urinary output. For example, the measuring device 3 or 3A may include a detection unit detecting blood included in urine, glucose included in urine, a specific weight of urine, urobilinogen included in urine, urinary protein, or the like. The detection unit may be provided in a manner of being attachable/detachable with respect to the housing 10 of the measuring device 3. The detection unit is connected to the control unit 14 to be able to communicate with each other.

From another viewpoint, the present invention provides a measuring device acquiring information related to urine stored in a urine bag. The measuring device includes a weighing unit configured to measure a mass value of the urine bag; a calculation unit configured to calculate at least one of a voided volume and a urinary output per unit time on the basis of the mass value weighed by the weighing unit; a transmission unit configured to transmit urinary output information related to at least one of the voided volume and the urinary output per unit time calculated by the calculation unit; a power source unit configured to supply power to the weighing unit, the calculation unit, and the transmission unit upon reception of supply of power from a power source outlet; and a first battery configured to supply power to the weighing unit, the calculation unit, and the transmission unit when power is not supplied from the power source unit.

In addition, the embodiment of the present invention may include a second battery configured to supply at least power for memory back-up when power is not supplied from the power source unit and the first battery.

### REFERENCE SIGNS LIST

1 Measuring system
3, 3A Measuring device
5 Management device
12 Weighing unit
14 Control unit (calculation unit, acquisition unit)
16 Touch panel display (display unit)
18 Communication unit (transmission unit)
20 Power source unit
22 First battery
23 Second battery
100 Urine bag

1. A measuring device acquiring information related to urine stored in a urine bag, the measuring device comprising:
a weighing unit configured to measure a mass value of the urine bag;
a calculation unit configured to calculate at least one of a voided volume and a urinary output per unit time on the basis of the mass value weighed by the weighing unit;
a transmission unit configured to transmit urinary output information related to at least one of the voided volume and the urinary output per unit time calculated by the calculation unit; and
a first battery configured to supply power to the weighing unit, the calculation unit, and the transmission unit;
a display unit configured to be able to display the urinary output information;
a control unit configured to control contents of display of the display unit; and
an acquisition unit configured to acquire a state of the first battery,
wherein the control unit causes the display unit to display that a residual quantity of the first battery has decreased when the residual quantity becomes equal to or smaller than a threshold.

2. (canceled)

3. (canceled)

4. The measuring device according to claim 1 further comprising:
a determination unit configured to determine anuria, oliguria, and polyuria on the basis of the urinary output information and a threshold set in advance,
wherein the transmission unit transmits notification information when the determination unit determines anuria, oliguria, or polyuria.

5. The measuring device according to claim 1 further comprising:
a second battery configured to supply at least power for memory back-up when power is not supplied from the first battery.

6. A measuring system comprising:
the measuring device according to claim 1; and
a management device configured to acquire information related to urine acquired by the measuring device.
